(19) United States
(12) Patent Application Publication
(10) Pub. No.: US 2026/0249054 A1
(43) Pub. Date: Aug. 27, 2026

(54) COIL REINFORCED SUPERELASTIC GUIDEWIRE

(71) Applicant: Abbott Cardiovascular Systems Inc., Santa Clara, CA (US)

(72) Inventor: Robert Charles Hayzelden, Murrieta, CA (US)

(73) Assignee: Abbott Cardiovascular Systems Inc., Santa Clara, CA (US)

(21) Appl. No.: 19/659,821

(22) Filed: Apr. 27, 2026

Related U.S. Application Data
(63) Continuation of application No. 16/861,064, filed on Apr. 28, 2020.

(51) Int. Cl. A61M 25/09

(52) U.S. Cl. CPC A61M 25/09; A61M 2025/09083; A61M 2025/09091; A61M 2025/09141; A61M 2025/09175

(57) ABSTRACT

A guidewire formed from a superelastic material has a proximal section and a distal section. In order to improve torque and pushability in the proximal section, a first wire is wound clockwise onto the proximal section to form a first coil and a second wire is wound counterclockwise onto the first coil to form a second coil.

### BACKGROUND

The application relates to guidewires configured for intraluminal application in medical procedures, and methods of their manufacture. More specifically, the application relates to guidewires that possess varying properties of flexibility and torsional stiffness along their length, and methods for making them.

Guidewires have long been known and used in the art of minimally invasive medical practice. Guidewires are typically used in conjunction with catheters in a procedure under which a placement catheter may first be threaded into the vasculature of a patient to a desired location using known techniques. A lumen within the placement catheter permits the physician to insert a guidewire through the catheter to the same location. Thereafter, when the physician may need to sequentially place a second, or third, or even a fourth catheter to the same location, it is a simple matter to withdraw the catheter while leaving the guidewire in place. After this action, second, third, and fourth etc. catheters may be sequentially introduced and withdrawn over the guidewire that was left in place. In other techniques, a guidewire may be introduced into the vasculature of a patient without the assistance of a placement catheter, and once in position, catheters may be sequentially inserted over the guidewire as desired.

It is typical that best medical practice for anatomical insertion requires a guidewire that has behavioral characteristics that vary along its length. For example, under some conditions, the distal end of the guidewire may be required to be more flexible than the proximal end so that the distal end may more easily be threaded around the more tortuous distal branches of the luminal anatomy. Further, the proximal end of the guidewire may be required to have greater torsional stiffness than the distal end because, upon rotation of the guidewire, the proximal end must carry all the torsional forces that are transmitted down the length of the guidewire, including what is required to overcome cumulative frictional losses.

While navigating a guidewire through tortuous anatomy during intervention procedures there is always a device compromise made to sacrifice push and torque transmission to the distal end to gain navigational and durability traits. Nitinol is often used to aid the physician in navigation and durability, however, the material properties of nitinol are not optimal for torque or push due to the superelastic nature of the material. The present invention solves the problems encountered by prior art guidewires formed from nitinol or other superelastic guidewires.

### SUMMARY OF THE INVENTION

In one embodiment, a single length of superelastic wire, preferably nitinol wire, alters the torque transmission, durability and pushability by reinforcing the proximal portion of the guidewire. The reinforcement can be accomplished by means of a coil or braided member placed over the core material and secured in place by means of a jacket. The outer coils or braid maybe constructed by means of a subassembly and slid onto the core, or maybe wound directly onto the core as a finished product. The outer jacket can be applied by means of dip coating, coextruding, or a shrink tube placed over the coils and secured in place. In one embodiment, the coils can be constructed in multiple configurations including multifilar, counter-wound multifilar, and materials of construction can include stainless steel or another material depending on the user needs for a specific application. Counter winding the coils will provide optimal torque in both the clockwise and counterclockwise directions. The coil wire (wrapped wire) cross-section can also be a specific profile, such as round, rectangular, or I-beam shaped, to optimize mechanical properties and torque transmission. Utilizing counter-rotated coils on the guidewire proximal end increases torquability in the proximal section while allowing a more compliant and flexible distal section for navigating tortuous vessels. Utilization of a braided or stranded coil over the core material also allows the outer jacket, once applied, to mimic the profile of the underlying structure providing an uneven surface which would both minimize surface contact (due to a decrease in resistance while in a tortuous bend) and potentially aid the physician in gripping and torqueing the device while navigating the guidewire into position.

In another embodiment, a guidewire includes an elongated core wire formed from a superelastic material. The elongated core wire has a proximal section and a distal section. A first wire is wound in a clockwise direction onto the proximal section of the elongated core wire to form a first coil, and a second wire is wound in a counterclockwise direction onto the first coil to form a second coil. A polymer cover is placed over the second coil and the proximal section of the elongated core wire to attach the first coil and the second coil to the elongated core wire.

### BRIEF DESCRIPTION OF THE DRAWINGS

FIG. 1 is an elevational view, partially in section, depicting a guidewire having counter-wound multifilar coils on the proximal section.

FIG. 2A is an enlarged, partial elevational view of the proximal section of the guidewire of FIG. 1, depicting a first multifilar wire being wound in a clockwise direction onto the proximal section.

FIG. 2B is an enlarged, partial elevational view of the proximal section of the guidewire of FIG. 2A, depicting the first multifilar wire forming first multifilar coils wound in a clockwise direction onto the proximal section

FIG. 3 is an enlarged, partial elevational view of the proximal section of FIG. 2B, depicting a second multifilar wire being wound in a counterclockwise direction onto the first multifilar coil to form a second multifilar coil.

FIG. 4 is an enlarged, partial elevational view of the first multifilar coil and the second multifilar coil being covered by a polymer cover.

FIG. 5 is an enlarged, partial elevational view, in section, depicting a first wire coil and a second wire coil being covered by a polymer cover.

### DETAILED DESCRIPTION OF THE PREFERRED EMBODIMENTS

While navigating a guidewire through tortuous anatomy during intervention procedures there is always a device compromise made to sacrifice push and torque transmission to the distal end to gain navigational and durability traits. Nitinol is often used to aid the physician in navigation and durability, however, the material properties of nitinol are not optimal for torque or push due to the superelastic nature of the material. The concept is to use a single length of superelastic wire or hollow tubing, such as nitinol, and alter the torque transmission, durability, and pushability, by reinforcing the proximal section of the guidewire.

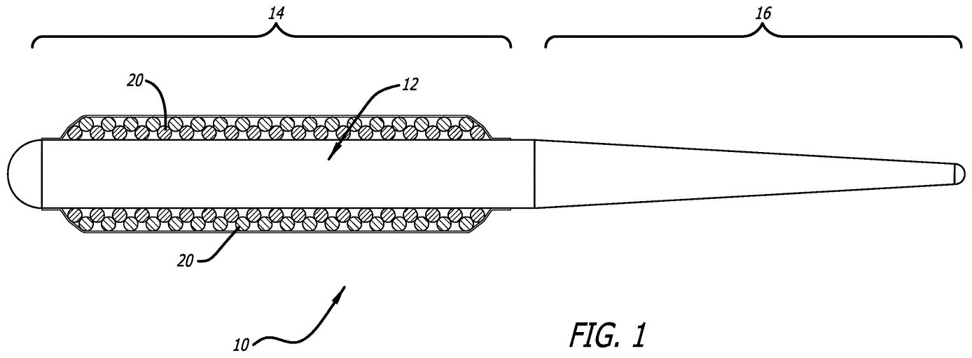

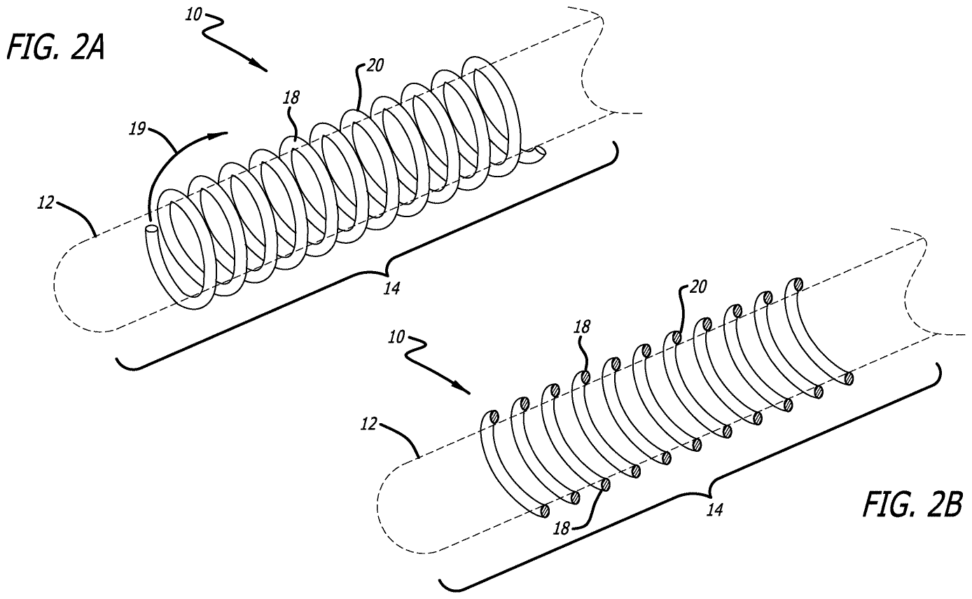

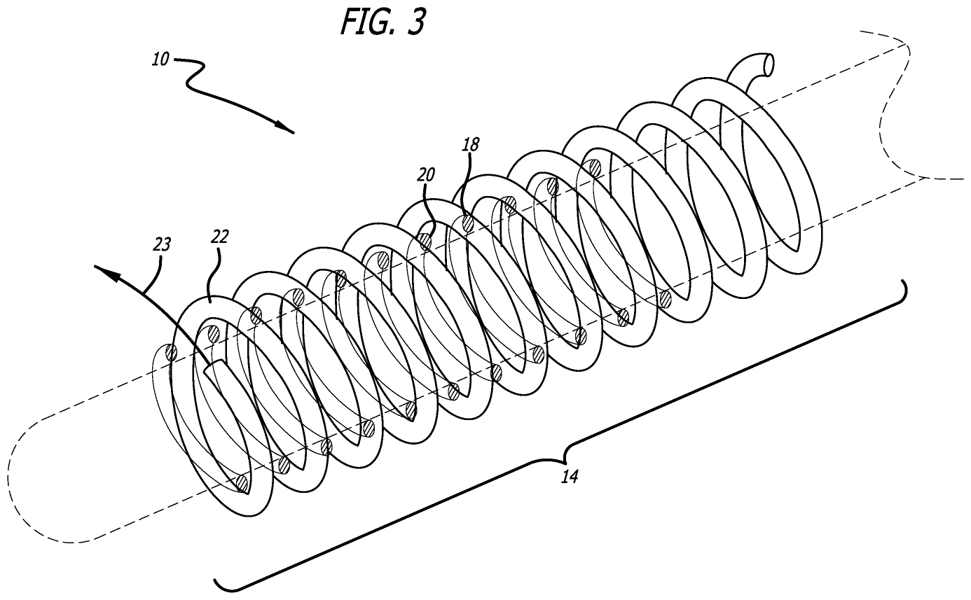

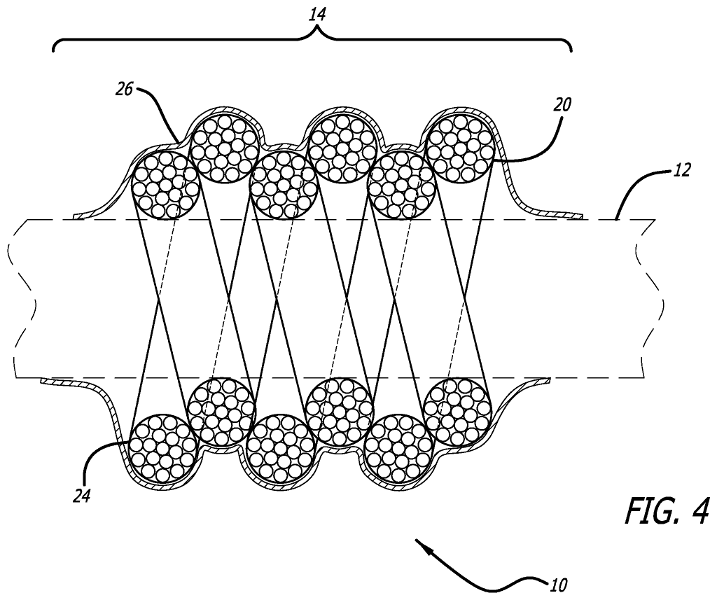

In one embodiment, as shown in FIGS. 1-4, a guidewire 10 includes an elongated core wire 12 formed from a superelastic material. The elongated core wire 12 has a proximal section 14 and a distal section 16. A first multifilar wire 18 is wound in a clockwise direction 19 onto the proximal section 14 of the elongated core wire 12 to form a first multifilar coil 20 (FIG. 2A), and a second multifilar wire 22 is wound in a counterclockwise direction 23 onto the first multifilar coil 20 to form a second multifilar coil 24 (FIG. 3). As shown in FIG. 4, a polymer cover 26 is placed over the second multifilar coil 24 and the proximal section 14 of the elongated core wire 12 to attach the first multifilar coil 20 and the second multifilar coil 24 to the elongated core wire 12. The polymer cover 26 can be applied by means of dip coating, coextruding, or a shrink tube placed over the coils 20, 24 and secured in place. Counter winding the coils 20, 24 will provide optimal torque in both the clockwise 19 and counterclockwise 23 directions. The transverse cross-section of the multifilar wires 18, 22 can also be a specific profile, such as round, rectangular, I-beam shaped, helical hollow strand, or stranded coil element/filament to optimize mechanical properties and torque transmission. Utilizing counter-wound coils 20, 24 on the guidewire proximal section 14 increases torquability in the proximal section 14 while allowing a more compliant and flexible distal section 16 for navigating tortuous vessels. Utilization of the multifilar coils over the proximal section also allows the polymer cover 26, once applied, to mimic the profile of the underlying structure (the multifilar coils 20, 24) providing an uneven surface which would both minimize surface contact (due to a decrease in resistance while in a tortuous bend) and potentially aid the physician in gripping and torqueing the device while navigating the guidewire into position. As an alternative to winding the first multifilar wire 18 and the second multifilar wire 22 onto the proximal section 14, the multifilar coils 20, 24 can be formed as subassemblies and advanced over the proximal section 14 and then secured in place by the polymer cover 26. The multifilar wires 18, 22 can be formed from a metallic material including stainless steel, titanium, cobalt chromium, tungsten, nitinol, platinum and silver.

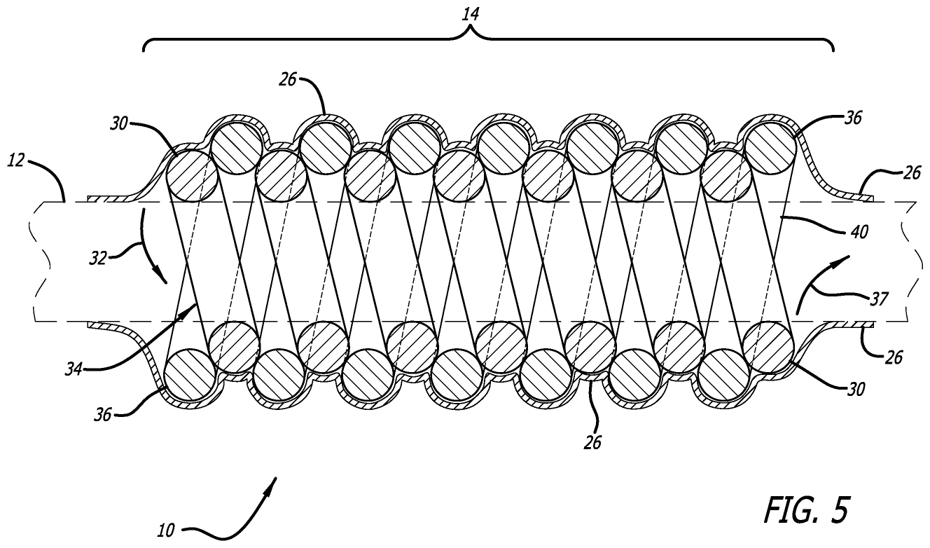

In another embodiment, as shown in FIG. 5, a guidewire 10 includes an elongated core wire 12 formed from a superelastic material similar to that shown in FIG. 1. The elongated core wire 12 has a proximal section 14 and a distal section not shown. A first wire 30 is wound in a clockwise direction 32 onto the proximal section 14 of the elongated core wire 12 to form a first coil 34, and a second wire 36 is wound in a counterclockwise direction 37 onto the first coil 34 to form a second coil 40. A polymer cover 26 is placed over the second coil 40 and the proximal section 14 of the elongated core wire 12 to attach the first coil 34 and the second coil 40 to the elongated core wire 12. The polymer cover 26 can be applied by means of dip coating, coextruding, or a shrink tube placed over the coils 34, 40 and secured in place. Counter winding the coils 34, 40 will provide optimal torque in both the clockwise 32 and counterclockwise 37 directions. The transverse cross-section of the first wire 30 and the second wire 36 can also be a specific profile, such as round, rectangular, I-beam shaped, or other known cross-sectional shapes, to optimize mechanical properties and torque transmission. Utilizing counter-rotated coils 34, 40 on the guidewire proximal section 14 increases torquability in the proximal section 14 while allowing a more compliant and flexible distal section for navigating tortuous vessels. Utilization of a single wire coil such as second coil 40 over the core material also allows the polymer cover 26, once applied, to mimic the profile of the underlying structure providing an uneven surface which would both minimize surface contact (due to a decrease in resistance while in a tortuous bend) and potentially aid the physician in gripping and torqueing the device while navigating the guidewire into position.

In all of the embodiments disclosed herein, a portion of or all of the elongated core wire 12 can be formed from a hollow tubing. The hollow tubing can improve guidewire performance, especially the torque and pushability performance. Application of the coils disclosed herein is the same for the hollow tubing as that described for the elongated core wire 12.

Guidewire lengths are well known in the art and can range from 180 cm to 300 cm for coronary artery applications, to much shorter lengths for other applications. Importantly, the proximal section 14 may be substantially longer than the distal section 16 of the elongated core wire 14. For example, for a standard 180 cm long guidewire 10, the proximal section can range from 95 cm to 180 cm, and preferably range from 165 cm to 175 cm.

The guidewire 10 preferably is formed from any superelastic material known in the art, and more preferably formed from nitinol. Guidewire 10 can be formed from other metal alloys including stainless steel, titanium, and cobalt chromium.

The polymer cover 26 can be formed by any polymer well known in the art for use with guidewire, catheters, and stents.

1-19. (canceled)

20. A guidewire, comprising:
an elongated core wire formed from a superelastic material;
the elongated core wire having a proximal section extending from a proximal end of the elongated core wire toward a distal end of the elongated core wire, and a tapered distal section of greater flexibility than the proximal section extending distally from a distal end of the proximal section;
a first multifilar wire wound in a first direction onto only the proximal section of the elongated core wire to form a first multifilar coil;
a second multifilar wire wound in a direction opposite to the first direction onto the first multifilar coil to form a second multifilar coil; and
a polymer cover with proximal and distal ends disposed over the second multifilar coil and secured at both ends to the proximal section of the elongated core wire to attach the first multifilar coil and the second multifilar coil to the proximal section of the elongated core wire to increase torqueability in the proximal section while allowing a more flexible distal section.

21. The guidewire of claim 20, wherein the first multifilar coil and the second multifilar coil are open pitch coils with gaps between adjacent windings, the first multifilar coil and the second multifilar coil being axially offset and arranged relative to one another such that the first multifilar coil engages the second multifilar coil in the gaps between adjacent windings of the second multifilar coil and the second multifilar coil engages the first multifilar coil in the gaps between adjacent windings of the first multifilar coil to define a combined coil assembly with an uneven outer profile, wherein the polymer cover surrounds the combined coil assembly and contacts both the first multifilar coil and the second multifilar coil to engage the uneven outer profile defined by the combined coil assembly and at least partially embed the combined coil assembly within the polymer cover.

22. The guidewire of claim 21, wherein the polymer cover is coextruded onto the combined coil assembly to form a continuous layer of uniform thickness that surrounds the combined coil assembly and retains the uneven outer profile of the combined coil assembly along an outer surface of the polymer cover.

23. The guidewire of claim 20, wherein the polymer cover extends only over the proximal section of the elongated core wire.

24. The guidewire of claim 20, wherein no portion of the first multifilar coil and no portion of the second multifilar coil covers or contacts the tapered distal section or any portion of the elongated core wire distal of the tapered distal section.

25. The guidewire of claim 20, wherein the proximal section of the elongated core wire has a uniform diameter.

26. The guidewire of claim 20, wherein the elongated core wire has an overall length extending from the proximal end to the distal end, and the proximal section of the elongated core wire is longer than the distal section and extends over a majority of the overall length from the proximal end.

27. The guidewire of claim 20, wherein the superelastic material forming the elongated core wire is taken from a group of materials including NiTi, CuZnAl and Co-Cr-N.

28. A guidewire, comprising:
an elongated core wire formed from a superelastic material;
the elongated core wire having a proximal section extending from a proximal end of the elongated core wire toward a distal end of the elongated core wire, and a tapered distal section of greater flexibility than the proximal section extending distally from a distal end of the proximal section;
a first wire wound in a first direction onto only the proximal section of the elongated core wire to form a first coil and a second wire wound in a direction opposite to the first direction onto the first coil to form a second coil; and
a polymer cover with proximal and distal ends disposed over the second coil and secured at both ends to the proximal section of the elongated core wire to attach the first coil and the second coil to the proximal section of the elongated core wire to increase torqueability in the proximal section while allowing a more flexible distal section.

29. The guidewire of claim 28, wherein the first coil and the second coil are open pitch coils with gaps between adjacent windings, the first coil and the second coil being axially offset and arranged relative to one another such that the first coil engages the second coil in the gaps between adjacent windings of the second coil and the second coil engages the first coil in the gaps between adjacent windings of the first coil to define a combined coil assembly with an uneven outer profile, and wherein the polymer cover surrounds the combined coil assembly and contacts both the first coil and the second coil to engage and conform to the uneven outer profile defined by the combined coil assembly and at least partially embed the combined coil assembly within the polymer cover.

30. The guidewire of claim 29, wherein the polymer cover is coextruded onto the combined coil assembly to form a continuous layer of uniform thickness that surrounds the combined coil assembly and retains the uneven outer profile of the combined coil assembly along an outer surface of the polymer cover.

31. The guidewire of claim 28, wherein the polymer cover extends only over the proximal section of the elongated core wire.

32. The guidewire of claim 28, wherein no portion of the first coil and no portion of the second coil covers or contacts the tapered distal section or any portion of the elongated core wire distal of the tapered distal section.

33. The guidewire of claim 28, wherein the proximal section of the elongated core wire has a uniform diameter.

34. The guidewire of claim 28, wherein the elongated core wire has an overall length extending from the proximal end to the distal end, and the proximal section of the elongated core wire is longer than the distal section and extends over a majority of the overall length from the proximal end.

35. A guidewire, comprising:
an elongated hollow tubing formed from a superelastic material;
the elongated hollow tubing having a proximal section extending from a proximal end of the elongated hollow tubing toward a distal end of the elongated hollow tubing, and a tapered distal section of greater flexibility than the proximal section extending distally from a distal end of the proximal section;
a first multifilar wire wound in a first direction onto only the proximal section of the elongated hollow tubing to form a first multifilar coil;
a second multifilar wire wound in a direction opposite to the first direction onto the first multifilar coil to form a second multifilar coil; and
a polymer cover with proximal and distal ends disposed over the second multifilar coil and secured at both ends to the proximal section of the elongated hollow tubing to attach the first multifilar coil and the second multifilar coil to the proximal section of the elongated hollow tubing to increase torqueability in the proximal section while allowing a more flexible distal section.

36. The guidewire of claim 35, wherein the first multifilar coil and the second multifilar coil are open pitch coils with gaps between adjacent windings, the first multifilar coil and the second multifilar coil being axially offset and arranged relative to one another such that the first multifilar coil engages the second multifilar coil in the gaps between adjacent windings of the second multifilar coil and the second multifilar coil engages the first multifilar coil in the gaps between adjacent windings of the first multifilar coil to define a combined coil assembly with an uneven outer profile, wherein the polymer cover surrounds the combined coil assembly and contacts both the first multifilar coil and the second multifilar coil to engage and conform to uneven outer profile defined by the combined coil assembly and at least partially embed the combined coil assembly within the polymer cover.

37. The guidewire of claim 36, wherein the polymer cover is coextruded onto the combined coil assembly to form a continuous layer of uniform thickness that surrounds the combined coil assembly and retains the uneven outer profile of the combined coil assembly along an outer surface of the polymer cover.

38. The guidewire of claim 35, wherein the polymer cover extends only over the proximal section of the elongated hollow tubing.

39. The guidewire of claim 35, wherein no portion of the first multifilar coil and no portion of the second multifilar coil covers or contacts the tapered distal section or any portion of the elongated hollow tubing distal of the tapered distal section.
